(19) United States
(12) Patent Application Publication
(10) Pub. No.: US 2020/0038433 A1
(43) Pub. Date: Feb. 6, 2020

(54) AQUEOUS COMPOSITION DERIVED FROM SEAWATER AND SEAWEED

(71) Applicant: YS LAB, QUIMPER (FR)

(72) Inventors: ROXANE FAGON, SAINT YVI (FR); MELODY DUTOT-WOLF, LA VARENNE SAINT HILAIRE (FR); CAROLINE TANTER, QUIMPER (FR); MARC HEMON, PLOUER SUR RANCE (FR)

(21) Appl. No.: 16/338,470

(22) PCT Filed: Sep. 26, 2017
(86) PCT No.: PCT/FR2017/052590

§ 371 Date: Mar. 30, 2019

(30) Foreign Application Priority Data
Oct. 3, 2016 (FR) 16 59484

(51) Int. Cl. A61K 31/737; A61K 36/03; A61K 33/20; A61K 33/06; A61K 33/00; A61K 9/00

(52) U.S. Cl. CPC A61K 31/737; A61K 36/03; A61K 33/20; A61K 9/0048; A61K 33/00; A61K 9/0014; A61K 33/06

(57) ABSTRACT

An aqueous composition for the stimulation of the immune system for the purpose of preventing at least one of the diseases provoked by the invasion of one or several pathogenic agents through the skin, the eye or a mucous membrane. The aqueous composition is obtained from seawater and includes a sulphated polysaccharide contained in a seaweed.

### RELATED APPLICATIONS

This application is § 371 application of PCT/FR2017/052590 filed Sep. 26, 2017, which claims priority from French Patent Application No. 16 59484 filed Oct. 3, 2016, each of which is incorporated herein by reference in its entirety

### FIELD OF THE INVENTION

This invention relates to a composition with a seawater base, intended in particular for the stimulation of the immune system with the purpose of preventing outside aggressions.

### BACKGROUND OF THE INVENTION

The physical defenses of the organism are the skin, the mucous membranes, the eyelashes and villi of the ciliated cells as well as the mucus and fluids that line most of the mucous membranes. These defenses make it possible to mechanically block the penetration of an exogenous agent in the organism and/or favour the evacuation thereof, in particular through the batting of the eyelashes.

The chemical and cellular defenses of the organism make use of several actors in several steps. In a first step at the beginning of the infection, the inflammatory response is triggered in a non-specific manner to activate the phagocytes and the macrophages which will digest the pathogenic agent after secretion of cytokines and other mediators of inflammation. In a second step, if the first line of defense was not enough to remove the pathogenic agent, the leukocytes assisted by the macrophages initiate the immune response in order to specifically recognise the pathogenic agent and to digest it. In certain cases, the organism does not succeed in fighting the invasion and a pathology develops. At the level of the ear-nose-throat sphere, in case of failure of the immune system, rhinitis, sinusitis and laryngitis can develop.

The first recommendations to keep the immune system reactive in case of an aggression are to maintain a healthy lifestyle: do not smoke, exercise regularly, limit the consumption of alcohol, get enough sleep, wash hands frequently, etc. The diet also plays a role in the maintaining of the immune system; for this reason, dietary supplements are available on the market intended to “boost the immune system”. These are mainly dietary supplements based on vitamins (A, B, C, D and E) and/or trace elements (selenium, zinc). However, no sufficiently exhaustive study has shown the efficacy of such dietary supplements. Other dietary supplements with a probiotic base make it possible to improve the operation of the digestive system and as such the assimilation of the nutrients essential for the synthesis of vitamins (B and K especially), but also to restore the commensal flora.

In terms of prevention, in order to block the development of certain serious infections against which the organism cannot fight, medicine proposes vaccination. Anti-infectious vaccination consists in introducing an antigen that is derived from or similar to the infectious agent, in order to encourage the organism to trigger an immune response. The vaccines can be attenuated or inactivated viruses (in order to prevent viral pathology), bacteria or subunit antigens. The immune reaction thus triggered makes it possible to memorise the antigen in order to allow the acquired immunity, during an actual contamination, to be activated faster.

Moreover, solutions are known that implement sulphated polysaccharides and more particularly fucans for the treatment of pathologies that make use of the defenses of the immune system. Such fucans are described in application WO2010/133359. Application WO2010/133359 describes the use of fucans involved in the good operation of the immune system with a positive impact on the health of an intestinal flora of a foetus during gestation.

### OBJECT AND SUMMARY OF THE INVENTION

There is a genuine need as it stands to improve the results obtained thanks to fucans for example by activating their effect thanks to a synergy of action with other elements. In order to fill in the lacks from prior art, the purpose of this invention is to propose a composition that makes it possible to intervene upstream before the entry and/or the proliferation of the pathogenic agents by stimulating the innate immunity.

An object of this invention relates to an aqueous composition for the stimulation of the immune system by stimulating the innate immunity for the purpose of preventing at least one of the diseases provoked by the invasion of one or several pathogenic agents by the skin, the eye or a mucous membrane obtained from 20 to 60% of filtered seawater, given as a percentage by weight in relation to the total mass of the composition and comprising a sulphated polysaccharide contained in a seaweed.

### BRIEF DESCRIPTION OF THE DRAWINGS

Other advantages, goals and features of the present invention emerge from the description that is made, for explanatory and in no way limiting purposes, regarding the appended drawings, in which:

FIGS. 1-2 show graphical evaluations of secretion of the antimicrobial peptide β-defensin-2 from the three Ascophyllum nodosum extracts tested;

FIGS. 3-4 show graphical evaluations of the inflammatory response with TNF-α with and without LPS;

FIG. 5 shows graphical evaluations of the inflammatory response with IL-1β with and without LPS;

FIG. 6 shows graphical measurements of the induction of the autophagy on macrophage after 1-hour incubation and 24 hours post-incubation;

FIG. 7 shows graphical measurements the secretion of the antimicrobial peptide β-defensin-2 by the macrophages;

FIG. 8 shows graphical comparison of the inflammatory response with an inflammatory cytokine (IL-1β) and an anti-inflammatory cytokine (IL-10);

FIG. 9 shows graphical evaluations of human pulmonary epithelial cell infected by a virus;

FIG. 10 are photos of ciliated epithelial cells incubated with a composition of the claimed invention and with a solution of isotonic sodium chloride; and

FIG. 11 shows a diagrammatical cross-section of a filtration unit.

### DETAILED DESCRIPTION OF THE EMBODIMENTS

In the framework of the invention the expression “contained in a seaweed” means that the composition comprises seaweed either entirely or partially, or that the composition includes a seaweed extract that comprises said sulphated polysaccharide, or that the sulphated polysaccharide used is a synthetic molecule which is identical to the natural molecule contained in seaweed.

In the framework of the invention the expression “obtained from seawater” means that the composition includes seawater, or that it contains transformed seawater.

Immunostimulation has for purpose to reinforce natural immunisation. The macrophages, first line of defense in the immunity response, have a dichotomous role: they trigger an inflammatory process that must then be inhibited in order to allow for the return to a physiological situation. These apparently contradictory effects reflect the complexity of the immune reaction. It is therefore entirely possible for an active ingredient to be immunostimulating and anti-inflammatory, it all depends on the mechanisms at play. Such effects have moreover been revealed in the framework of the study of Curcuma longa: Chandrasekaran et al. Pharmacognosy Res. 2013 April; 5(2): 71-79. This was also able to be observed in the framework of garlic extract: Ishiwada et al. J. Nutr. 2006; 136 (3 Suppl): 816S-820S; Keiss et al., J. Nutr. 2003; 133 (7): 2171-2175. Likewise, in the framework of the study of quercetine: Liao et al. J. Agric. Food. Chem. 2014 Apr. 2; 62(13):2872-80.

The inventors have unexpectedly shown that the composition according to the invention has immunostimulating effects by increasing the secretion of antimicrobial peptide qualified as β-defensin-2, by increasing the autophagy capacity of the cells, and by increasing the secretion of IL-10. The composition also has anti-inflammatory properties by inhibiting the secretion of TNFα and of IL-1β stimulated by LPS of E. Coli. Furthermore, it is interesting to note that in the absence of bacterial inflammatory stress (induced by the LPS), the composition according to the invention stimulates the immune response by slightly increasing the secretion of TNFα and of IL-1β (see the black rods of FIGS. 4 and 5).

Table 1 shows the constituent elements of seawater as well as their theoretical proportion, expressed in parts per million:

TABLE 1

| Element | ppm |
|---|---|
| Chlorine, Cl | 19,500 |
| Sodium, Na | 10,770 |
| Magnesium, Mg | 1,290 |
| Sulphur, S | 905 |
| Calcium, Ca | 412 |
| Potassium, K | 380 |
| Bromine, Br | 67 |
| Carbon, C | 28 |
| Nitrogen, N | 11.5 |
| Strontium, Sr | 8 |
| Oxygen, O | 6 |
| Boron, B | 4.4 |
| Silicon, Si | 2 |
| Fluorine, F | 1.3 |
| Argon, Ar | 0.43 |
| Lithium, Li | 0.18 |
| Rubidium, Rb | 0.12 |
| Phosphorus, P | 0.06 |
| Iodine, I | 0.06 |
| Barium, Ba | 0.02 |
| Molybdenium, Mo | 0.01 |
| Arsenic, As | 0.0037 |
| Uranium, U | 0.0032 |
| Vanadium, V | 0.0025 |
| Titanium, Ti | 0.001 |
| Zinc, Zn | 0.0005 |
| Nickel, Ni | 0.00048 |
| Aluminium, Al | 0.0004 |
| Cesium, Cs | 0.0004 |
| Chromium, Cr | 0.0003 |
| Antimony, Sb | 0.00024 |
| Krypton, Kr | 0.0002 |
| Selenium, Se | 0.0002 |
| Neon, Ne | 0.00012 |
| Manganese, Mn | 0.0001 |
| Cadmium, Cd | 0.0001 |
| Copper, Cu | 0.0001 |
| Tungsten, W | 0.0001 |
| Iron, Fe | 0.000055 |
| Xenon, Xe | 0.00005 |
| Zirconium, Zr | 0.00003 |
| Bismuth, Bi | 0.00002 |
| Niobium, Nb | 0.00001 |
| Thallium, Tl | 0.00001 |
| Thorium, Th | 0.00001 |
| Hafnium, Hf | 7 × 10−6 |
| Helium, He | 6.8 × 10−6 |
| Beryllium, Be | 5.6 × 10−6 |
| Germanium, Ge | 5 × 10−6 |
| Gold, Au | 4 × 10−6 |
| Rhenium, Re | 4 × 10−6 |
| Cobalt, Co | 3 × 10−6 |
| Lanthanum, La | 3 × 10−6 |
| Neodymium, Nd | 3 × 10−6 |
| Lead, Pb | 2 × 10−6 |
| Silver, Ag | 2 × 10−6 |
| Tantalum, Ta | 2 × 10−6 |
| Gallium, Ga | 2 × 10−6 |
| Yttrium, Y | 1.3 × 10−6 |
| Mercury, Hg | 1 × 10−6 |
| Cerium, Ce | 1 × 10−6 |
| Dysprosium, Dy | 9 × 10−7 |
| Erbium, Er | 8 × 10−7 |
| Ytterbium, Yb | 8 × 10−7 |
| Gadolinium, Gd | 7 × 10−7 |
| Praseodymium, Pr | 6 × 10−7 |
| Scandium, Sc | 6 × 10−7 |
| Tin, Sn | 6 × 10−7 |
| Holmium, Ho | 2 × 10−7 |
| Lutetium, Lu | 2 × 10−7 |
| Thulium, Tm | 2 × 10−7 |
| Indium, In | 1 × 10−7 |
| Trebium, Tb | 1 × 10−7 |
| Palladium, Pd | 5 × 10−8 |
| Samarium, Sm | 5 × 10−8 |
| Tellurium, Te | 1 × 10−8 |
| Europium, Eu | 1 × 10−8 |
| Radium, Ra | 7 × 10−11 |
| Protactinium, Pa | 5 × 10−11 |
| Radon, Rn | 6 × 10−16 |

The concentrations expressed in Table 1, are expressed as average values, which can vary according to the geographical location of the sea, and even the pumping location of the seawater.

The seawater at the base of the composition according to the invention, constitutes a reservoir of various chemical elements, present in a more or less substantial concentration. The physical chemical properties of seawater are dues to a balance between anions and cations. These elements behave as a set of biomolecules that interact with each other. In the framework of the invention, the synergy of action is done not only between these various ions, but also with sulphated polysaccharides present in the composition.

The inventors have entirely unexpectedly discovered that such a composition integrating a combination of ingredients coming from products from the sea, which ingredients come both from substances dissolved in seawater and from seaweed which have their growth take place in seawater, made it possible to prevent the development of diseases linked to the incorporation and proliferation of pathogenic agents in the organism. The target pathogens are mainly viruses or bacteria or allergenic agents. The composition according to the invention is intended for prophylactic treatment in order to act upstream by helping the organism to guard against the harmful effects of such pathogenic agents by stimulating the innate immunity.

This composition according to the invention stimulates the innate immunity, i.e. it allows for an activation of the defenses against pathogenic agents such as those mentioned hereinabove, without the body of the subject having necessarily been put into contact with said agents beforehand.

It has been demonstrated that the composition according to the invention is particularly advantageous through the fact that a biological response is induced right from the first application. An effect in vitro is observed on live cells after only one hour of being putting into contact.

In addition, the measured effect is memorised by the cells and remains remnant even after the elimination of the solution. Consequently, the biological action is therefore preventive.

Furthermore, the biological action is observed for different cell types, which results in an action possible at different locations of the organism by a local application directly on cells. The action of preparing the organism to defend itself is carried out by various complementary mechanisms allowing for a protection with regards to the various types of pathogenic agents (bacteria, viruses, allergens, fungal microorganisms).

The composition according to the invention comprises 20 to 60%, and preferably 35 to 55%, even 40 to 50% of filtered seawater given as a percentage by weight in relation to the total mass of the final composition obtained. The filtered seawater is a seawater that is treated by carrying out a passage through at least one decontaminating filter; the diameter of the pores of said filter is advantageously less than 1 μm.

The seawater is advantageously a Breton seawater selected preferably in the town of Penmarch in the département of Finistère, in the Bay of Saint-Brieuc in the département of Côtes-d'Armor, or in the Bay of Saint-Malo in the département of Ille and Vilaine.

The seawater used is selected for its privileged location, which conditions and/or imposes:
an absence of industries in the vicinity with a healthy environment;
a sampling site that is not enclosed: constant renewal of water;
current in the zone that favour the renewal of the water; and
a water with quality that is recognised as satisfactory by specialised bodies, such as IFREMER and DDASS (departmental directorate of and health and social affairs).

The composition according to the invention advantageously comprises at least one water selected from purified water, distilled water and demineralised water in a content from 40 to 80%, preferably 45 to 65, even from 50 to 60%. The purity of the dilution water used to adjust the concentrations is closely controlled in order to prevent the adding of undesirable elements that can pollute or modify the physical chemical properties of the medium.

Advantageously, the sulphated polysaccharide is a fucan. A definition of fucans in terms of the invention is given in application WO1999032099.

The sulphated polysaccharide is advantageously extracted from a brown seaweed. The brown seaweed is preferably selected from one of the species: Ascophyllum nodosum, Fucus vesiculosus, and Undaria pinnatifida.

Preferably, the brown seaweed is Ascophyllum nodosum. The sulphated polysaccharides extracted from Ascophyllum nodosum have demonstrated a strong synergy of action in combination with the elements contained in seawater for the stimulation of the antimicrobial peptide β-defensins-2, involved in the innate immunity which can be considered as a first line of defense of the organism against microbial attacks.

The sulphated polysaccharide is advantageously taken in a content from 0.01 to 0.5%, preferably 0.05 to 0.4%, even from 0.09 to 0.25%.

The sulphated polysaccharide advantageously has a mass from 2 to 21 kDa (kilodalton), preferably 4 to 17.5 kDa, even 5 to 15 kDa. The tests have shown that among the polysaccharides and in particular fucans, those of 4, 8 and 20 kDa made it possible to obtain a very good antimicrobial activity. The best results were obtained with fucans of 8 kDa.

Advantageously, the concentration of the elements in the final composition, present in ionic form, is such that:

| | |
|---|---|
| Cl (g/L) | 5-15 |
| Na (g/L) | 2.5-7 |
| Mg (g/L) | 0.45-0.70 |
| Ca (g/L) | 0.10-0.30 |
| K (g/L) | 0.10-0.30 |

Advantageously, the concentration of the elements of the substances in the final composition according to the invention is such that:

| | |
|---|---|
| Cl (g/L) | 7.9-10.1 |
| Na (g/L) | 4.3-5.3 |
| Mg (g/L) | 0.51-0.63 |
| Ca (g/L) | 0.16-0.22 |
| K (g/L) | 0.15-0.23 |
| Total sugars (g/L) | 0.25-0.55 |

Advantageously the content of the following substances in the composition according to the invention is such that:

| | |
|---|---|
| Total sulphates (g/Kg) | 1.0-1.8 |

The concentrations in the composition used for a biological action are low for a strong cellular response, which reveals a high coefficient of efficiency of this natural marine complex.

The composition shows to be completely harmless to patients at the concentrations described, making it able to be used in the general population but also from a paediatric standpoint.

The composition has also shown perfect tolerance for the mucous membranes, even the most delicate such as ciliated epithelium of the nasal mucous membrane. Indeed, the study of the cytotoxicity of the composition according to the invention made it possible to evaluate its high tolerance for the uses considered. Various evaluation tests of the cytotoxicity on several types of cells as well as a study of the tolerance in humans have been conducted in order to better ascertain the tolerance of the composition according to the invention by the organism. An observation study in healthy subjects shows this tolerance by complying with the normal operation of the ciliated cells of the nasal mucous membrane.

Advantageously, the pH of the composition is 6.5 to 8.5. Preferably, the pH is from 6.9 to 8.1.

Advantageously, the osmolality measured at 25° C. is from 400 to 550 mOsmol/Kg. Preferably, the osmolality is from 450 to 500 mOsmol/Kg.

The osmolality is measured according to the method described in European Pharmacopeia 9.0 (ninth edition)—point 2.2.35. The conductivity is measured according to the method described in European Pharmacopeia 9.0—point 2.2.38.

The stimulation of the immunity of the composition according to the invention consists advantageously in a stimulation of the innate immunity.

The mucous membrane or membranes targeted by the preventive treatment according to the invention is, or are, selected from the nasal mucous membrane, the oral mucous membrane and the vaginal mucous membrane. Advantageously, the composition according to the invention relates to the prevention of the inflammatory states of the mucous membranes.

The composition according to the invention can be applied to any field that makes use of cellular physiology, in the organs, such as the eye or such as the organs of the ENT sphere—in particular the nasal mucous membranes—with the purpose of preventing affections such as rhinitis, nasal congestions, dry nose, sinusitis or bronchitis. The composition according to the invention can also be applied to the skin and to the vaginal mucous membranes, in particular in preparations for intimate hygiene.

This invention also relates to a method of preparing a composition such as described hereinabove in the framework of the invention, comprising the steps of:
a) pumping seawater;
b) optionally pretreating the seawater using a first natural or artificial filter;
c) filtering the optionally pretreated seawater through a second filter of which the average diameter of the pores is less than or equal to 5 μm;
d) carrying out another filtration through a third filter of which the average diameter of the pores is less than those of the second filter and is less than or equal to 0.45 μm;
e) diluting the filtered solution with purified water.

In step a), the seawater taken via pumping is optionally filtered (step b)) with the assistance, either of a natural filter such as for example sand, or of an artificial filter such as for example a grid adapted to remove large-size particles. The water is then advantageously stored in a pretreatment basin or tank. This seawater is then redirected to the pipes using a pump for the purpose of the filtration thereof.

The pretreated seawater is filtered in the steps c) and d) through two filters of which the average diameters are respectively from 5 μm to 1 μm (for c)), and less than 0.45 μM (for d)). For this step of filtration, the water is preferably introduced into a filtration unit that integrates pipes which advantageously include filters in the form of cylindrical cartridges. An example of a filtration unit is described hereinbelow in the experimental part.

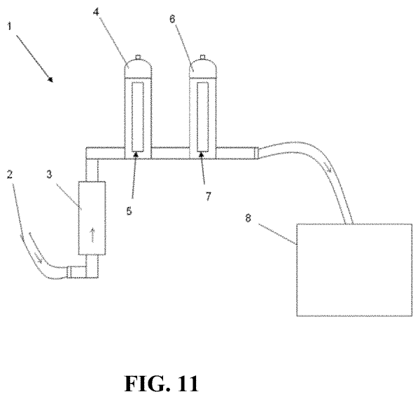

This invention shall be better understood when reading the experimental part which shall follow, of which the examples and the figures are given solely for the purposes of information and which cannot in any way be considered as limiting. In order to illustrate the results of the experimental part, FIG. 11 shows a diagrammatical cross-section of a filtration unit which makes it possible to filter the seawater before the dilution thereof in order to obtain the composition according to the invention.

### EXPERIMENTAL PART

### Preparation of a Composition According to the Invention Comprising 45% of Seawater (FIG. 11):

45 Kilograms of a seawater taken in Brittany via pumping is filtered using a hopper. The seawater thus taken is stored in a pretreatment basin. This seawater is then pumped using a pump.

The pretreated seawater is injected into the pipes of a filtration unit 1 shown in FIG. 11. The filtration unit 1 comprises an inlet 2 of seawater. This water inlet 2 is connected to a flowmeter 3 which is connected to the bottom portion of the casing 4 of a prefilter 5 (1 μm). The outlet of the prefilter 5 (still in the bottom portion of the casing 4) is connected to the bottom portion of the casing 6 of a filter 7 (0.22 μm) of which the outlet is connected to a pipe which is used to collect the water coming from the filtration in order to store it in a container 8.

The filtered solution is then supplemented with purified water and the seaweed extract is added.

### I. Test of the Immunological Efficacy of the Composition by the Study of Various Markers of Immunity Defenses

Various seaweed extracts, rich in fucans of different molecular weights, were compared.

Evaluation of the Secretion of the Antimicrobial Peptide β-Defensin-2

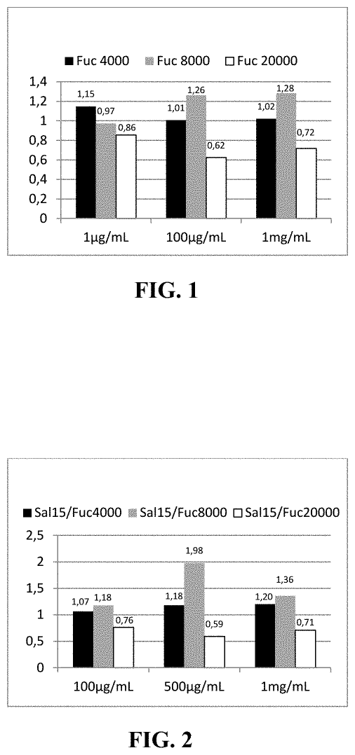

Three Ascophyllum nodosum extracts were tested: one rich in fucans de 4,000 Da (Fuc 4000, black rod in FIGS. 1 and 2), another rich in fucans de 8,000 Da (Fuc 8000, grey rod in FIGS. 1 and 2) and a last one rich in fucans of 20,000 Da (Fuc 20000, white rod in FIGS. 1 and 2). A Fucus vesiculosus (Fuc Fucus) extract and an Undaria pinnatifida (Fuc Undaria) extract, of which the molecular weight of the fucans is between 5,000 and 30,000 Da, were also tested. These samples are solubilised either in the culture medium RPMI containing 2.5% of foetal calf serum, 0.5% of penicillin/streptomycin and 1% of glutamine, or in seawater at 45% (Sal 15) at three concentrations (1 μg/mL, 100 μg/mL and 1 mg/mL). The samples are incubated with the macrophages for 1 h then the supernatants are recovered for the assay of the antimicrobial peptide β-defensin-2.

The salting-out of β-defensin-2 is measured on the macrophages using the ELISA (Enzyme-Linked Immunosorbent Assay) technique. This technique allows for the assay of proteins in a sample; in our case, the ELISA technique allows us to quantify the peptide β-defensin-2, released in the cell supernatant by the macrophages. The specific antigen of the β-defensin-2 is attached to a support, a 96-well microplate. The cell supernatant is deposited thereon and the β-defensin-2 can bind to its antigen. A primary antibody makes it possible to recognise the β-defensin-2, then a secondary antibody conjugated with an enzyme binds to the primary antibody. A colorimetric enzymatic reaction allows for the revelation of the antibody complex. The device used to quantify the absorbance is a cytometer adapted to microplates (Safire, TECAN).

The secretion of β-defensin-2 was evaluated by measuring the secretion factor which is listed on the ordinate axis in FIGS. 1 and 2.

The Ascophyllum nodosum extract rich in fucans with a molecular weight 8000 Da (Fuc 8000) is the one that is the most effective for stimulating the secretion of the antimicrobial peptide β-defensin-2.

These tests unambiguously demonstrate the synergy of action between the fucans and the filtered seawater in the stimulation of the innate immune system (at 1 μg/mL: ×0.97 in the culture medium versus ×1.18 in seawater, at 100 μg/mL: ×1.26 versus ×1.98 and at 1 mg/mL: ×1.28 versus ×1.36).

Evaluation of the Inflammatory Response with TNF-α

The salting-out de proinflammatory cytokines (TNF-α) is measured on the macrophages by the ELISA technique, explained hereinabove.

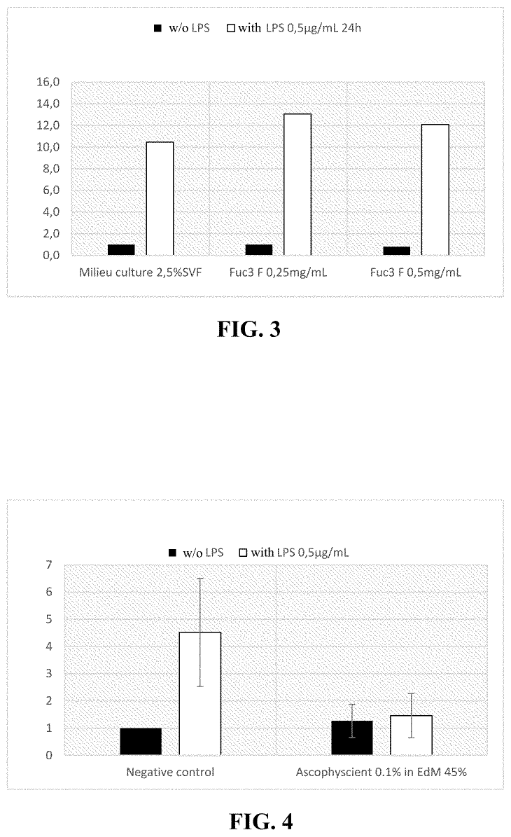

An Ascophyllum nodosum extract rich in fucans with an average molecular weight of 10,000 Da (Ascophyscient) and a Laminaria digitata extract rich in fucans with an average molecular weight of 520,000 Da (Fuc3 F) were tested for their capacity in reducing the secretion of proinflammatory cytokines (TNF-α) induced by a bacterial agent (white rods in FIGS. 3 and 4, versus the same samples without LPS: black rods), the LPS of Escherichia Coli for 24 hours at 37° C. They are solubilised either in the culture medium RPMI containing 2.5% of foetal calf serum, 0.5% of penicillin/streptomycin and 1% of glutamine, or in the seawater at 45% at the concentrations indicated in FIGS. 3 and 4, and incubated with the macrophages.

The secretion of proinflammatory cytokines was measured by the assay of TNF-α which is shown in FIGS. 3 and 4, with the scale of the ordinates represented in arbitrary units:
As can be seen when interpreting the results in FIG. 3, the Laminaria digitata (Fuc3 F) extract does not stimulate the production of proinflammatory cytokine TNF-α. It is not capable of blocking the production of cytokine induced by the LPS of Escherichia Coli. It therefore does not have an anti-inflammatory effect in relation to TNF-α.
On the other hand, if the same assay is carried out after incubation of the macrophages with the composition containing 0.1% of an Ascophyllum nodosum (Ascophyscient) extract rich in fucans with an average molecular weight 10 kDa dissolved in 45% of filtered seawater (composition Stimmuno_A), in this case, the secretion of TNF-α induced by the LPS is blocked (FIG. 4). The Ascophyllum nodosum extract rich in fucans with an average molecular weight of 10,000 Da (Ascophyscient) in combination with 45% of the filtered seawater therefore has a strong anti-inflammatory effect.

Evaluation of the Inflammatory Response with IL-113

The ELISA technique is also used here.

In order to confirm the control of the inflammatory reaction measured with TNF-α by the formulation, the assay of another proinflammatory cytokine, IL-1β, was therefore conducted.

As can be seen when interpreting the results shown in the FIG. 5 which shows the assay of IL-1β (arbitrary units on the ordinate axis), the Ascophyllum nodosum (Ascophyscient) dissolved in 45% of filtered seawater (composition Stimmuno_A) does not stimulate the production of proinflammatory cytokine IL-113. Consequently, the composition Stimmuno_A according to the invention with 0.1% of Ascophyllum nodosum and 45% of filtered seawater is capable of blocking the production of cytokines induced by the LPS of Escherichia Coli. which shows a control of the secretion of IL-1β in response to a bacterial stress.

Conclusion:

Ascophyllum nodosum is effective for decreasing the production of proinflammatory cytokines. The Ascophyllum nodosum extract rich in fucans with a molecular weight of 10,000 Da controls the inflammatory response contrary to the Laminaria digitata extract rich in fucans of 520,000 Da.

Other seaweed extracts and in particular Fucus vesiculosus and Undaria pinnatifida in filtered seawater were also tested, and made it possible to conclude that the best results were obtained with Ascophyllum nodosum.

Evaluation of the Autophagy and Revealing of the Synergy of Action

Other tests (part A. and B.) were also conducted, in order to reveal the effectiveness of the composition with regards to markers of cell defense that were studied after incubation of human cells with compositions containing 0.1-0.2% of an Ascophyllum nodosum (Ascophyscient) extract rich in fucans with an average molecular weight of 10 kDa dissolved in 45% of filtered seawater and filled to 100% with purified water (qsp): the autophagy and the secretion of the antimicrobial peptide β-defensin-2.

### Part A.

The composition Stimmuno_A (0.1% (m/v) of an Ascophyllum nodosum (Ascophyscient) extract rich in fucans with an average molecular weight of 10 kDa dissolved in 45% of filtered seawater and filled to 100% with purified water (qsp)) is put into contact with cells of the immune system (human macrophages) for 1 h, the formulation is removed then the induction of the autophagy is measured immediately or 24 h after. The autophagy is a catabolic process that makes it possible to maintain the cellular homeostasis. It is activated in case of cellular stress for the digestion of the pathogenic elements. In parallel, the supernatants are recovered in order to assay therein the antimicrobial peptide β-defensin-2 that the macrophages secrete in order to eliminate the microorganisms.

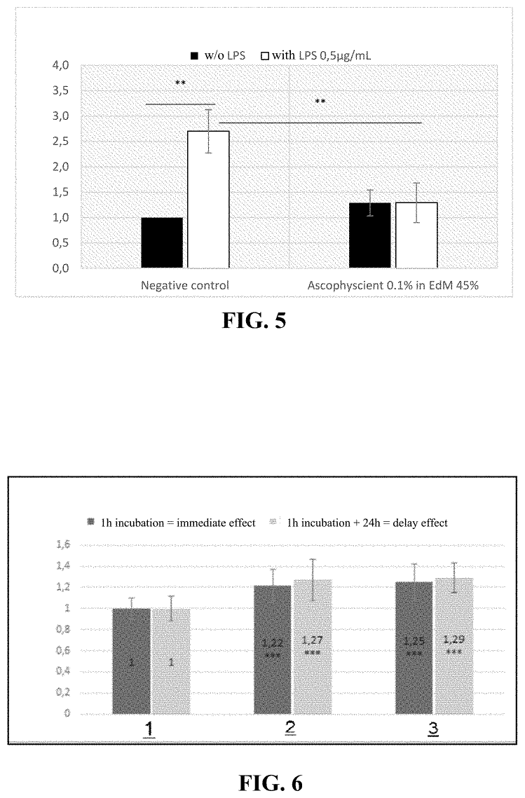

The results obtained are presented in FIG. 6 which shows the measurement of the autophagy on macrophage by cytometry (monodansylcadavrine test, MDC). During the formation thereof, the autophagosome—which consists in a sequestration compartment with a double membrane generated from a phagosome—incorporates in its membrane cytoplasmic components, of which MDC, fluorescent. After incubation with the samples, the supernatants were sampled for a later assay of the peptide β-defensin-2 and the cells are incubated with a solution of MDC at 0.05 mM (stock solution to be prepared in the DMSO at 50 mM) in the buffer PBS for 30 minutes at 37° C. After incubation, the sensor is removed and the cells are rinsed with PBS. The microplate is then read by the Safire-TECAN cytofluorimeter at the wavelengths of the MDC (λexcitation=380 nm, λemission=525 nm).

The solid grey rods on the left quantify the immediate effect measured after 1 h of incubation, the hashed rods on the right quantify the delayed effect after 1+24 h of incubation. The sample 1 corresponds to the negative control, the sample 2 (in the middle of FIG. 6) corresponds to the test conducted on the composition Stimmuno_A, and the sample 3 to the test conducted on a positive control. The positive control used here for the induction of the autophagy is a bacterial agent LPS of Escherichia Coli at 0.5 μg/mL.

The scale of the ordinates corresponds to the measured values of the modulation factor in relation to the negative control.

The three asterisks “***” in FIG. 6 that are mentioned on the rods respectively for the samples 2 and 3 correspond to a statistical significance of p<0.001 (probability that the difference is random) compared to the negative control (ANOVA, Dunnett test, GraphPad Prism 6 software).

Conclusion:

After incubation (1 h) with the composition Stimmuno_A, the autophagy is significantly increased in the macrophages whether immediately after removal of the composition or 24 h after. The composition Stimmuno_A makes it possible to stimulate the autophagy, a catabolic process that makes it possible to maintain the cellular homeostasis via cell digestion, as prevision of an aggression by pathogenic elements.

### Part B.

Comparative tests were conducted between:
a composition according to the invention, the composition Stimmuno_B (0.2% (m/v) of an Ascophyllum nodosum (Ascophyscient) extract rich in fucans with an average molecular weight of 10 kDa dissolved in 45% of filtered seawater and filled to 100% with purified water (qsp));
a composition AN: including only 0.2% (m/v) of an Ascophyllum nodosum (Ascophyscient) extract dissolved in the buffer PBS (saline phosphate buffer solution, commonly used in cell culture); and
a composition EDM: including filtered seawater containing 15 g/L in NaCl.

The results shown in FIG. 7 which compiles the measurements of the secretion of the antimicrobial peptide β-defensin-2 by the macrophages (ELISA technique); the scale on the ordinate axis corresponds to the measured values of the modulation factor of the secretion in relation to the buffer PBS alone: the sample 1 correspond to the PBS alone, the sample 2 (in the middle of FIG. 7) corresponds to the test conducted on the composition Stimmuno_B, the sample 3 to the test conducted on the composition AN, and the sample 4 to the test conducted on the composition EDM.

After incubation (1 h) with the composition Stimmuno_B the secretion by the macrophages of the antimicrobial peptide β-defensin-2 is increased 24 h after. On the other hand, the composition AN, as well as the composition EDM do not have any effect on the secretion of the antimicrobial peptide β-defensin-2. The composition Stimmuno_B makes it possible to stimulate the secretion of the antimicrobial peptide β-defensin-2, as prevision of an aggression by pathogenic elements.

Conclusion:

There is therefore a synergy of action between the components of the filtered seawater and the sulphated polysaccharides.

Evaluation of the Activity on Different Models with Different Stress Agents: Marker of Innate Immunity

A composition Stimmuno_C according to the invention was also tested on different cell models with different stress agents according to the same protocol: the product is put into contact with the cells for a short period of time (1 h) then the compositions are removed and the cells are stressed by the adding of a pathogenic toxic agent. At the time of the stress, the cells are therefore no longer in contact with the composition, however the inflammatory reaction is significantly inhibited: this means that the cells have metabolised and have memorised the signals triggered by the composition Stimmuno_C in order to thus be able to control the inflammatory response.

Composition Stimmuno_C:
Solution of 253 kg prepared with 116.4 kg of filtered seawater, 269.5 g of Ascophyllum nodosum extract rich in fucans (Ascophyscient), 136 g of purified water.
The characteristics of the solution obtained are:
PH: 7.2
Osmolality: 471 mosmol/Kg
Chloride expressed in NaCl: 14.6 g/I
Total sugars: 0.395 g/l (or total oses measured by the method of Dubois and a. (1956))

Immune cells (macrophages) were incubated for 1 h with on the one hand, the composition Stimmuno_C, and on the other hand the culture medium (MC 0%=negative control) then the solutions were removed and the cells incubated with LPS of Escherichia Coli in order to induce a bacterial inflammatory stress.

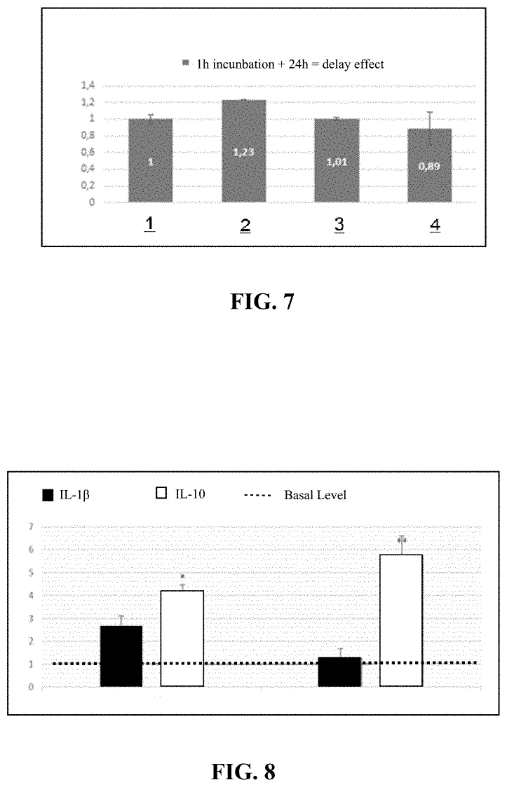

Two cytokines were assayed by the ELISA test in the cell supernatants: a proinflammatory cytokine, IL-1β (black rods in FIG. 8) and an anti-inflammatory cytokine, IL-10 (white rods in FIG. 8).

The results are shown in FIG. 8 which compiles the assays of cytokines after 1 h of incubation followed by 24 h in the LPS of Escherichia Coli on macrophage. The scale on the ordinate axis corresponds to the amplifying factors of secretion. The black dotted line represents the basal level of secretion without LPS which is 1.

When the cells have been incubated with the culture-negative control medium (to the left in FIG. 8), the LPS stimulates the secretion of IL-1β by a factor 2.7 and that of IL-10 by a factor 4.2 in relation to the basal level of secretion without LPS. The cells thus respond to the LPS by secreting an attack signal (IL-1β) and a defense signal (IL-10). When the cells have been incubated with the composition Stimmuno_C (on the right in FIG. 8), the LPS stimulates the secretion of IL-1β by a factor 1.3 and that of IL-10 by a factor 5.8. Consequently, the composition Stimmuno_C decreases the attack signal while still stimulating the defense signal induced by the LPS.

### Conclusion:

The inflammatory reaction is significantly inhibited: this means that the cells have metabolised and have memorised (memory effect) the signals triggered by the composition Stimmuno_C in order to thus be able to control the inflammatory response. These experimental results demonstrate the stimulation of the innate immunity (memory effect) by the composition according to the invention.

Evaluation of the Activity on Human Pulmonary Epithelial Cell Infected by a Virus

Pulmonary epithelial cells are taken from a human, and incubated in the presence of the composition Stimmuno_A. It is shown that the composition according to the invention dramatically decreases the expression of genes involved in the inflammatory response as well as the secretion of proinflammatory cytokines induced by the polyIC, viral stress agent.

Bronchial epithelial cells taken from a human were incubated for 1 h with NaCl 0.9% (utilised here as a negative control) or the composition Stimmuno_A, then the solutions were removed and the cells incubated with PolyIC in order to induce a viral inflammatory stress.

The expression of the gene of the proinflammatory cytokine CCL20 was assayed by RTqPCR. The expression of CCL20 is upregulated in patients afflicted with chronic rhinosinusitis.

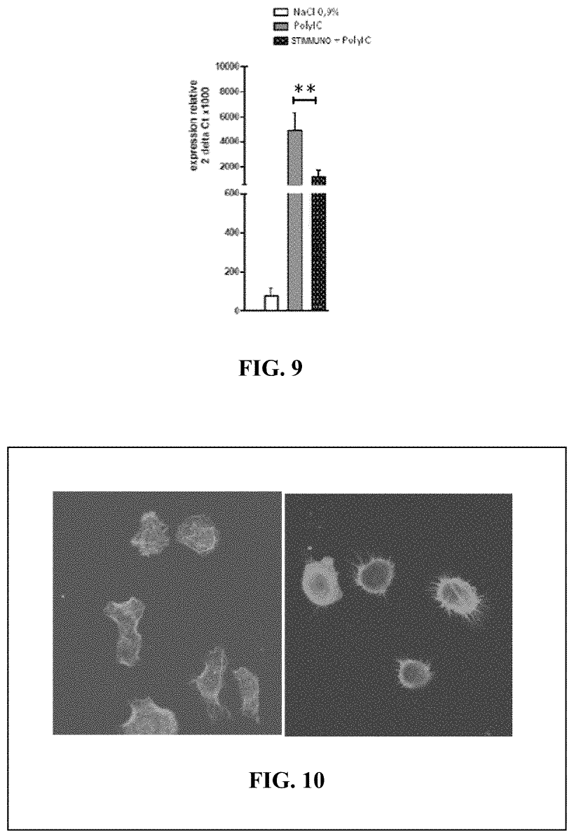

The results are shown in FIG. 9, which quantifies the expression of the gene of the protein CCL20 by the bronchial epithelial cells. The scale on the ordinate axis shows the values measured for the relative expression (2 delta Ct×1000).

The PolyIC stimulates the overexpression of the gene CCL20 (orange bar), but when the cells were incubated with the composition Stimmuno_A, the PolyIC stimulates the secretion of this gene much less (decrease by a factor 25). Consequently, the composition Stimmuno_A decreases the attack signal induced by the PolyIC.

### Conclusion:

The results show that on the pulmonary epithelial cells taken from a human, the composition Stimmuno_A dramatically decreases the expression of genes involved in the inflammatory response as well as the secretion of proinflammatory cytokines induced by the polyIC, viral stress agent.

As a general conclusion, the composition according to the invention prepares the cells of the organism to defend themselves in case of a bacterial or viral attack. The composition blocks and controls the inflammatory response which could induce various external aggressions. This defense applies to the various points of entry into the organism: nasal mucous membrane, oral mucous membrane, throat, eye, skin, vaginal mucous membrane, etc

II. Tests for Innocuity with Regards to the Cells of the Organism

The good tolerance of the composition containing 0.1% of an Ascophyllum nodosum (Ascophyscient) extract rich in fucans with an average molecular weight of 10 kDa dissolved in 45% of filtered seawater (composition Stimmuno_A) for the nasal epithelium was confirmed by the study of the morphology of the ciliated cells of the nasal epithelium. The vibrating cilia of these cells make it possible to eliminate the mucus. The maintaining of the cilia morphology is essential for the good operation of the nasal epithelium.

Protocol:

Human cells of the nasal epithelium were incubated either with isotonic sodium chloride at 0.9%, or with the composition for 1 h. The cytoskeleton of the cells was coloured by a fluorescent marker (green) and the cores with another fluorescent marker (blue).

FIG. 1 shows that the eyelashes are much more visible and longer when the ciliated epithelial cells are incubated with the composition according to the invention (shown on the photo on the right) in relation to the solution of isotonic sodium chloride (shown on the photo on the left).

It can therefore be concluded that the composition containing the Ascophyllum nodosum extract and 45% of filtered seawater is therefore perfectly tolerated by the human nasal epithelium.

1-14. (canceled)

15. An aqueous composition to stimulate an immune system by stimulating an innate immunity to prevent at least one disease provoked by an invasion of at least one pathogenic agent through at least one of a skin, an eye and a mucous membrane, the aqueous composition obtained from 20 to 60% filtered seawater and comprising a sulphated polysaccharide contained in a seaweed.

16. The aqueous composition according to claim 15, wherein the sulphated polysaccharide is a fucan.

17. The aqueous composition according to claim 15, further comprising at least one water selected from a purified water, a distilled water and a demineralised water in a content from 40 to 80%.

18. The aqueous composition according to claim 15, wherein the sulphated polysaccharide is extracted from a brown seaweed.

19. The aqueous composition according to claim 18, wherein the brown seaweed is Ascophyllum nodosum.

20. The aqueous composition according to claim 15, wherein the sulphated polysaccharide is in a content from 0.01 to 0.5%.

21. The aqueous composition according to claim 15, wherein the sulphated polysaccharide has a mass from 2 to 21 kDa.

22. The aqueous composition according to claim 15, wherein concentrations of elements of the aqueous composition are:

| | |
|---|---|
| Cl (g/L) | 5-15; |
| Na (g/L) | 2.5-7; |
| Mg (g/L) | 0.45-0.70; |
| Ca (g/L) | 0.10-0.30; and |
| K (g/L) | 0.10-0.30. |

23. The aqueous composition according to claim 15, wherein concentrations of elements of substances in the aqueous composition are:

| | |
|---|---|
| Cl (g/L) | 7.9-10.1; |
| Na (g/L) | 4.3-5.3; |
| Mg (g/L) | 0.51-0.63; |
| Ca (g/L) | 0.16-0.22; |
| K (g/L) | 0.15-0.23; and |
| Total sugars (g/L) | 0.25-0.55. |

24. The aqueous composition according to claim 15, wherein a pH is from 6.5 to 8.5.

25. The aqueous composition according to claim 15, wherein an osmolarity at 25° C. is from 400 to 550 mOsmol/Kg.

26. The aqueous composition according to claim 15, wherein said mucous membrane is a nasal mucous membrane, an oral mucous membrane or a vaginal mucous membrane.

27. The aqueous composition according to claim 15, to prevent inflammatory states of the mucous membrane.
